(19) United States
(12) Patent Application Publication
(10) Pub. No.: US 2024/0345422 A1
(43) Pub. Date: Oct. 17, 2024

(54) MELANOPSIN BLOCKER

(71) Applicant: Arizona Board of Regents on Behalf of the University of Arizona, Tucson, AZ (US)

(72) Inventors: Brooke Jordan Mason, Tucson, AZ (US); Fabian Fernandez, Tucson, AZ (US); Michael Grandner, Tucson, AZ (US); Stanley K. H. Pau, Tucson, AZ (US)

(21) Appl. No.: 18/292,183

(22) PCT Filed: Jul. 25, 2022
(86) PCT No.: PCT/US2022/038235

§ 371 Date: Jan. 25, 2024

Related U.S. Application Data
(60) Provisional application No. 63/225,848, filed on Jul. 26, 2021.

(51) Int. Cl. G02C 7/10

(52) U.S. Cl. CPC G02C 7/107

(57) ABSTRACT

Methods and devices are described that relate to spectral filters and associated eyewear that are specifically designed to block emissions of circadian-active blue light to reach the observer. An example wearable device includes one or more windows positioned to allow light from a light source to propagate toward a position of a wearer's eyes, and a spectral filter that comprises a coating positioned on one or more sections of the one or more windows. The spectral filter includes a multi-layer stack of dielectric material. The number and thicknesses of the layers are selected to provide designed transmission and blocking characteristics. The designed characteristics include a contiguous blocking region that blocks 98-100% of the circadian-active spectra and two contiguous transmission regions that transmit 80%-100% of the spectral content outside of the circadian-active spectra.

### CROSS-REFERENCE TO RELATED APPLICATION(S)

This application claims priority to the provisional application with Ser. No. 63/225,848 titled “MELANOPSIN BLOCKER,” filed Jul. 26, 2021. The entire contents of the above noted provisional application are incorporated by reference as part of the disclosure of this document

### TECHNICAL FIELD

The disclosed embodiments relate to eyewear and spectral filters.

### SUMMARY

Methods and devices are disclosed that, among providing other features and benefits, relate to spectral filters and associated eyewear that are specifically designed to block emissions of circadian-active blue light to reach the observer.

An example wearable device includes one or more windows positioned to allow light from a light source to propagate toward a position of a wearer's eyes, and a spectral filter that comprises a coating positioned on one or more sections of the one or more windows. The spectral filter includes a multi-layer stack of dielectric material with alternate high and low indices of refraction such that a layer having a high index of refraction is positioned above or below a layer having a low index of reflection, and a layer having a high index of refraction is positioned above or below a layer having a low index of reflection. The number of the layers and a thickness of each layer are selected to provide designed transmission and blocking characteristics to block circadian-active spectra while allowing spectral content outside of the circadian-active spectra to pass through the spectral filter. The designed transmission and blocking characteristics include a contiguous blocking region within 455-495 nm band of wavelengths with a tolerance to within at least ±5 nm, and two contiguous transmission regions, a first one of the contiguous regions extending below 455 nm and a second one of the contiguous transmission regions extending above 495 nm. The spectral filter is configured to block 98-100% of the spectral content in the contiguous blocking region and transmit 80%-100% of the spectral content in the contiguous transmission regions.

### BRIEF DESCRIPTION OF THE DRAWINGS

FIG. 1 illustrates example spectral characteristics of dye-based (left) and pigment-based (right) filters.

FIG. 2A illustrates the solar radiation spectrum.

FIG. 2B illustrates the radiation spectrum of an example light emitting diode (LED).

FIG. 2C illustrates the spectrum of an example halogen lamp.

FIG. 3 illustrates a typical spectral sensitivity of the human eye.

FIG. 4A illustrates a plot of the transmission spectra for a filter specifically designed to block certain circadian-active light in accordance with an example embodiment.

FIG. 4B illustrates a set of parameters associated with the filter of FIG. 4A.

FIG. 5A illustrates a plot of the transmission spectra for a filter specifically designed to block certain circadian-active light in accordance with another example embodiment.

FIG. 5B illustrates a set of parameters associated with the filter of FIG. 5A.

FIG. 6 illustrates an example eyewear that incorporates the melanopsin blocker filters of the disclosed technology.

### DETAILED DESCRIPTION

The timing of the brain's circadian clock is set by schedules of natural sunlight exposure. All physiological processes in the body are synchronized to the signals sent from the brain's clock as it interprets these environmental light patterns. As such, the clock's estimation of whether it is daytime (presence of light) or nighttime (near absence of light) has widespread impacts on the timing and organization of the sleep-wake cycle. Electric room lighting can interfere with the circadian clock's estimation of day versus night because the photoreceptors in the eye that send information to the clock are activated by both natural sunlight and electric room lighting. Electric lighting can thus interfere with sleep because humans are most biologically prepared to sleep at night not during the day. If the clock interprets a daytime signal when sensing electric light, it will delay the timing of sleep and will have more difficulty communicating with the rest of the body so that a consolidated period of rest is coordinated at night.

The photoreceptors that transmit light information to the brain's clock are most sensitive to certain parts of the light spectrum. One photoreceptor, melanopsin, is expressed by a subpopulation of cells in the eye called intrinsically photosensitive retinal ganglion cells (ipRGCs). They are most activated by light occurring between 455-495 nm, which is perceived as “blue light.” A second important photoreceptor that relays information to the clock is expressed by cone cells in the retina that are sensitive to mid-wavelength light occurring between 500-560 nm, which is perceived as “green light.” Though blue and green light are only partial pieces of the overall visible spectrum (400-800 nm), they have outsized effects on how the brain's clock measures light exposure and interprets for itself and for the rest of the body the timing and length of the day versus the timing and length of the night.

The circuitry that comprises the brain's clock is different from the circuitry that comprises the image-forming visual system (i.e., the system that allows us to see). Current lens technologies that seek to limit the sleep-disrupting effects of electric lighting at night, take advantage of this segregation by filtering out all incident light below ˜560 nm and allowing the rest through to enable visibility. This approach blocks ˜50% of circadian-active light, while maintaining approximately 80% visibility.

The disclosed embodiments, among other features and benefits, rely on interference filter configurations that are implemented in a wearable device (e.g., glasses, goggles, etc.) or used as a covering for a luminaire (e.g., a light source from a house lamp) that provide a precise and granular spectral behavior by precisely specifying what light is blocked versus that which is allowed to go through within that part of the range that is normally completely blocked up to 560 nm with current lens technologies. The disclosed technology can be implemented by designing a lens that maintains the visibility almost completely intact, while only blocking the most circadian-active blue light occurring between 455-495 nm (i.e., that which activates melanopsin). The result of this more precise targeting of circadian-active light is that about 75% the spectrum that is blocked by current lens technology between 400-560 nm would be freed up to maximize perceived visibility.

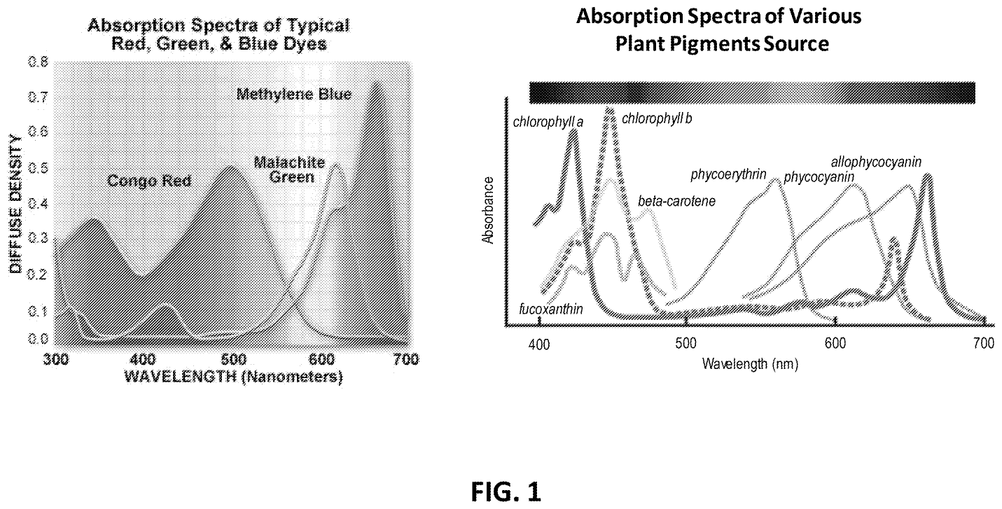

A large number of the existing systems rely on dye- or pigment-based filters that block or transmit a contiguous band of wavelengths but without a capability to selectively transmit or block narrower subbands within the larger contiguous band. Dye and pigment filters operate based on absorption of light by color dye and pigment embedded in a material such as polymer or sol-gel. The transmission spectrum of this type of filter has broad peaks shaped like a Gaussian function with linewidth equal to the inhomogeneous broadening of the materials. FIG. 1 illustrates example spectral characteristics of dye-based (left) and pigment-based (right) filters that exhibit this type of behavior. As also evident from FIG. 1, each spectrum includes a prominent peak with gradual fall off characteristics on both sides thereof. Thus the usefulness of these filters can be limited to applications where the desired spectral range happens to coincide with the spectral peak of the filter. But even then, the gradual falloff of the spectra can cause part of the desired spectrum to be filtered while allowing part of the undesired spectral content to seep through. In addition, the materials can be bleached under high light intensity, high temperature and/or corrosive environment.

Other types of filters include doped glass, semiconductor, metal, and metamaterial optical filters. Doped glass filters are made of a glass doped with a trace of impurity such as a metal and semiconductor nanocrystal, silver halides and cuprous ions. Semiconductor optical filters are made of semiconductor material with a transmission edge determined by the bandgap. Metal optical filters are made by depositing several layers of metal or metallic alloy made of rhodium, palladium, tungsten, nickel and chromium on a transparent substrate and are used extensively as neutral density filters. Metamaterial optical filters are made of micro- and nano-fabricated structures with dimensions of the order of or smaller than the operating wavelength. Another class of optical filters are tunable optical filters with transmission spectra that can be changed by temperature, electric and/or magnetic field. Examples of tunable optical filters are liquid crystal, Fabry-Perot and MEMS filters. These types of filters are generally bulky and have a lower transmission than non-tunable filters.

The disclosed embodiments rely on multi-layer dielectric interference filter configurations to enable the precise spectral shaping that is required for precise channeling of circadian-active green light. Multi-layer dielectric or dichroic filters operate by optical interference instead of absorption. These filters are made by depositing multiple layers of dielectric coating such as magnesium fluoride, zinc sulfide, cerium dioxide, titanium dioxide, silicon oxide, zirconium dioxide to name a few. Interference filters can be designed to transmit light of different wavelength band with sharp transmission edge, in contrast to the broad band spectrum of the dye and pigment filter. The transmission spectrum of this type of filter is generally dependent on the angle of the incident light, although designs can be made to minimize the angular variation.

Several types of interference filters are described that relate to the features of the disclosed embodiments: long-wave pass, short-wave pass, notch (minus, bandstop), and band pass interference filter. A long-wave pass interference filter can include a multilayer structure and can be described using the following shorthand notation:

$${[ \frac{H}{2} L \frac{H}{2} ]}^{s} .$$

In the above expression, H denotes a quarter-wave high-index layer having a thickness λ₀/4n_{H} and H/2 denotes half of a quarter-wave high-index layer, i.e., one-eighth of a wave λ₀/8n_{H}. L denotes a quarter-wave low-index layer having a thickness λ₀/4n_{L}; s is an integer that denotes the number of basic periods (i.e., how many times the basis structure of high-low-high is repeated), λ₀ is the reference wavelength (i.e., the center wavelength used to design the filter), and n_{H,L} represents the high or the low refractive index, depending on whether the H or L subscript is used. A short-wave pass interference filter can include a multilayer structure and can be described by the following notation that follows a similar convention as described above:

$${[ \frac{L}{2} H \frac{L}{2} ]}^{s} .$$

A bandpass filter is a combination of long-wave pass and short-wave pass filters, and allows only a particular spectral band (i.e., the passband) to be transmitted. A notch filter blocks a particular band of wavelengths (i.e., the notch) but allows the remaining spectral content to pass therethrough. A notch filter can be implemented by using a multilayer structure, represented by the following notation:

[αLβH]^{s}αL.

In the above expression, α and β are numbers chosen for the location and width of the notch filter. For example, a notch filter with a reference wavelength at 550 nm and bandwidth of about 100 nm can be implemented using the multilayer structure represented by:

[1.68L0.30H]⁵⁹1.68L.

In the above example, α=1.68 and β=0.30.

One key advantage of the disclosed embodiments is the selective transmission and blocking of different wavelengths of light to match the photo receptor sensitivity of the human retina with high efficiency that maintains a high visibility. To this end, the coating on the lens that is part of the eyewear is specifically designed to elicit a particular biological response. To meet these requirements, the optical lens with the coating must satisfy two efficiency conditions: transmission efficiency and illumination efficiency. The transmission efficiency of a color filter can be described as:

$${η}_{T} = \frac{{\int }_{{λ}_{1}}^{{λ}_{2}} d λ T ( λ )}{{\int }_{{λ}_{3}}^{{λ}_{4}} d λ T ( λ )} .$$

In the above expression, η_{T} is the transmission efficiency; λ₁ and λ₂ are the lower and upper wavelengths, respectively, of the transmission band; λ₃ and λ₄ are the lower and upper wavelengths, respectively, of the incident illumination; and T(λ) is the filter transmission spectrum. Interference filters with sharp transition edge and low transmission ripple are used to achieve high η_{T}.

In addition, the illumination efficiency of a color filter can be described as:

$${η}_{i} = \frac{{\int }_{{λ}_{1}}^{{λ}_{2}} d λ S ( λ ) T ( λ ) ρ ( λ )}{{\int }_{{λ}_{3}}^{{λ}_{4}} d λ S ( λ ) ρ ( λ )} .$$

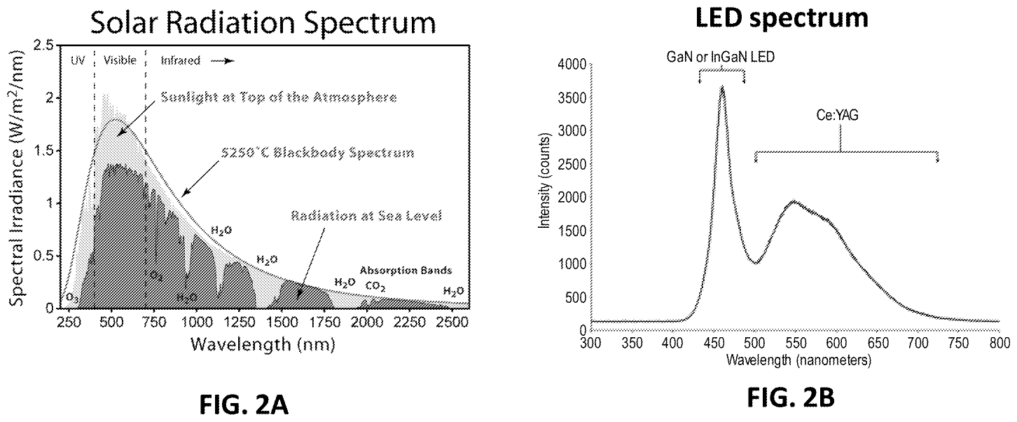

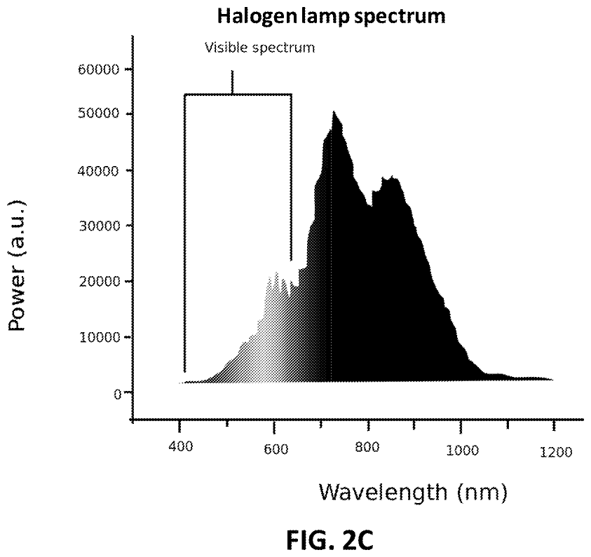

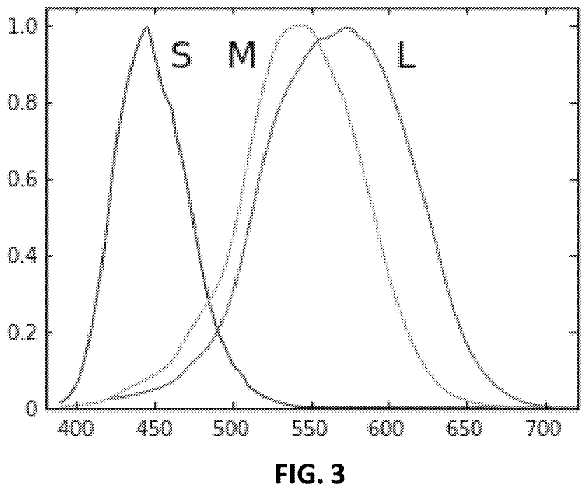

In the above expression, ηᵢ is the illumination efficiency; λ₁ and λ₂ are the lower and upper wavelengths, respectively, of the transmission band; λ₃ and λ₄ are the lower and upper wavelengths, respectively, of the incident illumination; S(λ) is the illumination spectrum; T(λ) is the filter transmission spectrum; and ρ(λ) is the human eye sensitivity. S(λ) can be the spectrum of the sun, a light emitting diode, a halogen lamp, a fluorescent lamp, or another source of illumination. Example spectra of some of the above sources are presented in FIGS. 2A to 2C. In particular, FIG. 2A illustrates the solar radiation spectrum; FIG. 2B illustrates the radiation spectrum of an example LED; and FIG. 2C illustrates the spectrum of an example halogen lamp. FIG. 3 illustrates a typical spectral sensitivity of the human eye, and in particular, the normalized responsivity spectra for S-, M- and L-cone cells. The illumination efficiency of the lens must be high so that light visibility is not critically reduced during day and night.

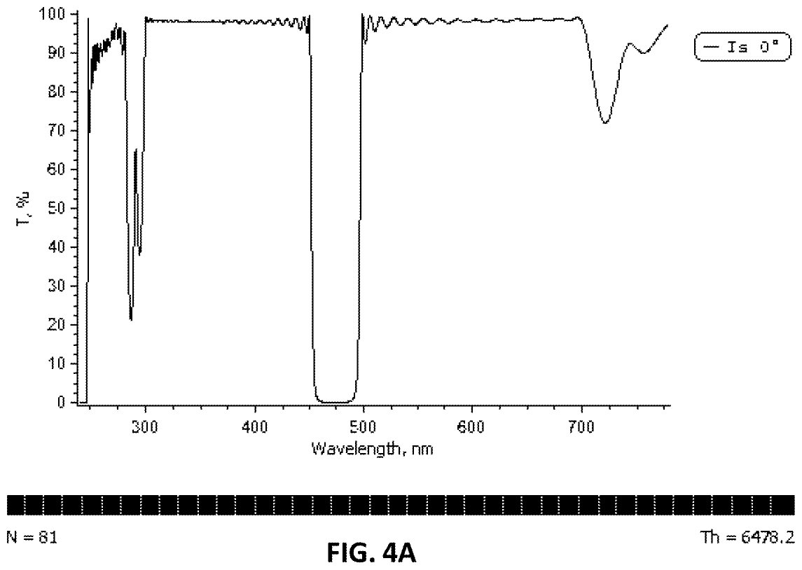

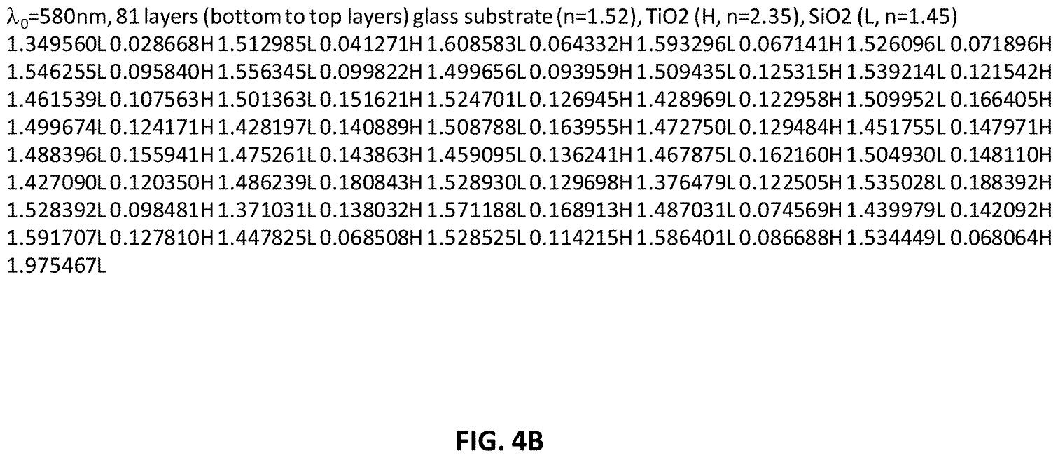

FIG. 4A illustrates a plot of the transmission spectra for a filter specifically designed to block certain circadian-active light in accordance with an example embodiment. In particular, the filter in FIG. 4A complete blockage (in some embodiments, with nearly 0% transmission) of spectral content in the band 455-495 nm (±2 nm), while allowing the spectral content in the bands 300-455 nm and 495-700 nm (±5 nm) to be transmitted (in some embodiments, with nearly 100% transmission). It should be noted that the spectral tolerance (listed in parenthesis) is selected to block all, or almost all, of the spectral content in the 455-495 nm band with high precision. The filter in FIG. 4A includes 81 dielectric layers stacked on top of a glass substrate (n=1.52), with high and low alternated layers that include TiO₂ (H, n=2.35) and SiO₂ (L, n=1.45), respectively. The thicknesses of the layers (using λ₀=475 nm as the reference wavelength) are listed in FIG. 4B. The transmission (or blockage) characteristics of the spectral bands can be modified by changing the number of layers in the design. For example, in some embodiments, the filter is designed to provide a blockage between 98-100%. In other filters, the blockage can be 95-98%. Yet in other designs, the blockage is 80-95%. In some example embodiments, the transmission is in the range 98-100%. Generally, the characteristics of the spectral bands can be fine-tuned by adding more layers to the design at the expense of increasing the cost of the filters.

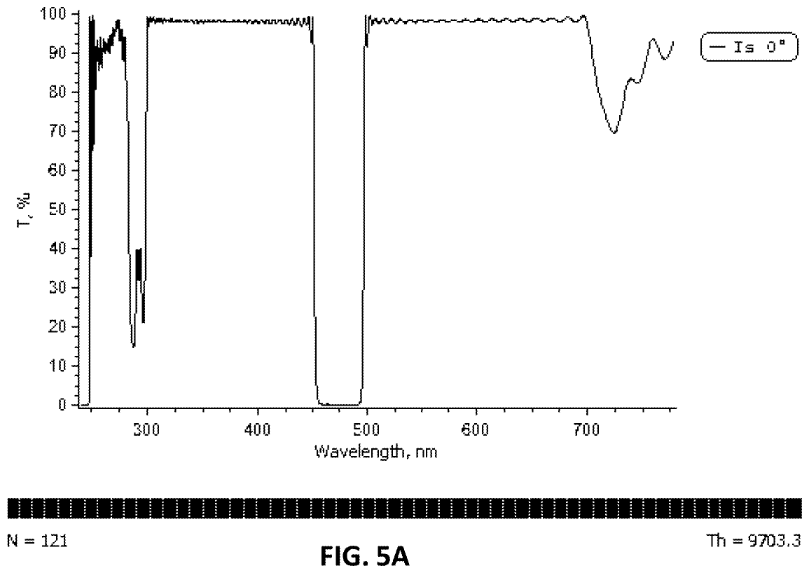

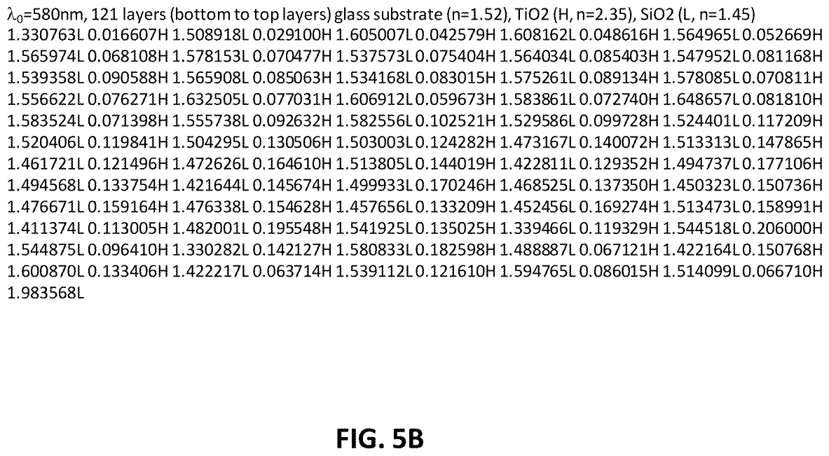

FIG. 5A illustrates a plot of the transmission spectra for a filter specifically designed to block certain circadian-active wavelengths in accordance with an alternate embodiment. In particular, the filter in FIG. 5A includes a high transmission region (in some embodiments, with close to 100% transmission capability) in bands 300-455 nm and 495-700 nm (±5 nm), while blocking (i.e., with nearly 0% transmission) the spectral contents in the range 455-495 nm (±2 nm). The filter has 121 dielectric layers (designed with a reference wavelength λ₀=580) stacked on top of a glass substrate with thicknesses that are listed in FIG. 4B. Compared to the FIG. 4A filter, the filter in FIG. 5A has smaller ripple and sharper band edge transitions. These characteristics are obtained by using larger number of dielectric layers. Generally, a filter with more relaxed tolerances and lower number of layers is easier to manufacture, i.e., potentially has higher yield, and cost less.

Notably, the filters in FIGS. 4A and 5A block nearly 100% of light all spectral content in the range 455-495 nm, which contains a large portion of the circadian-active illumination, while allowing the remaining spectral content to pass through with high transmission. As a result, the visibility of the surrounding environment and objects is substantially preserved while, at the same time, a large portion of circadian-disruptive illumination is blocked from reaching the viewer.

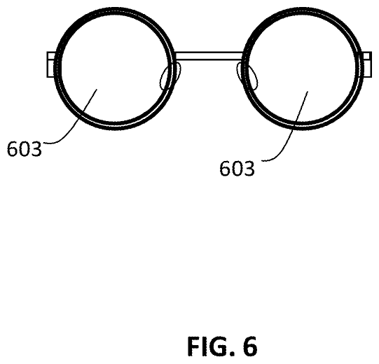

The disclosed filters can be implemented as part of specialized glasses or goggles (e.g., virtual reality goggles). FIG. 6 illustrate a pair of example glasses that includes a pair of lenses 603 that can be coated with the disclosed filters. In some implementations, the glasses can include opaque side shields or blocks (not shown) to prevent side illumination to reach the eye. In some implementations, the side shields can be transparent and include filters with similar transmission and blocking characteristic as those on the lenses 603. The disclosed filters can be implemented as a coating provided on other types of eyewear, such as goggles. For instance, the wearable device can include a unitary transparent window that can be coated uniformly, or at particular locations thereon, with the disclosed filters having transmission and blockage characteristics at precisely tailored bands of the spectrum. For example, the particular locations of coatings can be selected to affect light that reaches the user's eyes at approximately normal angles. In another example, the coatings' locations and areal extent can be chosen to filter the light that reaches the user's eyes at both normal and inclined angles. In yet another example, the filters can be made detachable to existing eye wear, for example by small magnets or by screw-in adapter.

One aspect of the disclosed embodiments relates to a wearable device that includes one or more windows positioned to allow light from a light source to propagate toward a position of a wearer's eyes, and a spectral filter that comprises a coating positioned on one or more sections of the one or more windows. The spectral filter includes a multi-layer stack of dielectric material with alternate high and low indices of refraction such that a layer having a high index of refraction is positioned above or below a layer having a low index of reflection, and a layer having a high index of refraction is positioned above or below a layer having a low index of reflection. The number of the layers and a thickness of each layer are selected to provide designed transmission and blocking characteristics to block circadian-active spectra while allowing spectral content outside of the circadian-active spectra to pass through the spectral filter. The designed transmission and blocking characteristics include a contiguous blocking region within 455-495 nm band of wavelengths with a tolerance to within at least ±5 nm, and two contiguous transmission regions, a first one of the contiguous regions extending below 455 nm and a second one of the contiguous transmission regions extending above 495 nm. The spectral filter is configured to block 98-100% of the spectral content in the contiguous blocking region, and transmit 80%-100% of the spectral content in the contiguous transmission regions.

In one example embodiment, the contiguous blocking region extends from 455 nm to 495 nm, the first contiguous transmission region extends from 300 nm to 455 nm, and the second contiguous transmission region extends from 495 nm to at least 700 nm, all with a ±5 nm tolerance. In another example embodiment, each layer with the high index of refraction includes titanium dioxide (TiO₂) and has a 2.35 index of refraction, each layer with the low index of refraction includes silicon dioxide (SiO₂) and has a 1.45 index of refraction, and the multi-layer stack includes 81 layers. In still another example embodiment, the contiguous blocking region extends from 455 nm to 495 nm, the first contiguous transmission region extends from 300 nm to 455 nm, and the second contiguous transmission region extends from 495 nm to at least 700 nm, all with a ±2 nm tolerance. In yet another example embodiment, each layer with the high index of refraction includes titanium dioxide (TiO₂) and has a 2.35 index of refraction, each layer with the low index of refraction includes silicon dioxide (SiO₂) and has a 1.45 index of refraction, and the multi-layer stack includes 121 layers.

According to one example embodiment, the one or more windows include two lenses, and the spectral filter is formed as the coating on each of the lenses. In another example embodiment, the wearable device is a pair of goggles, the one or more windows forms a unitary window, and the spectral filter is formed as the coating the unitary window. In yet another example embodiment, the wearable device is a pair of goggles, the one or more windows forms a unitary window, and the spectral filter is formed as the coating on the two or more sections of the unitary window. In still another example embodiment, the one or more windows are made of glass or plastic.

In another example embodiment, the spectral filter is removably attached to the one or more windows. According to another example embodiment, the light source is one or more of: an atmospheric light source, a light emitting diode (LED), a halogen lamp, or a fluorescent lamp. In another example embodiment, the wearable device further includes an anti-reflection coating positioned on one side of the one or more windows.

In another example embodiment, the wearable device is a pair of goggles, the one or more windows form a unitary window, the spectral filter is formed as the coating on the two or more sections of the unitary window, and locations and areal extents of the two or more sections of the unitary window are selected to allow light propagating at normal angles to pass through the spectral filter and reach the position of the wearer's eyes. In yet another example embodiment, the locations and areal extents of the two or more sections of the unitary window are selected to allow light propagating at inclined angles to pass through the spectral filter and reach the position of the wearer's eyes. In another example embodiment, the goggles include opaque side shields positioned to prevent side illumination from reaching the position of the viewer's eyes.

Another aspect of the disclosed embodiments relate to a spectral filter for use in an eyewear for restoring circadian rhythm that includes a multi-layer stack coating on a substrate, the multi-layer stack including a plurality of layers of dielectric material with alternate high and low indices of refraction such that a layer having a high index of refraction is positioned above or below a layer having a low index of reflection, and a layer having a high index of refraction is positioned above or below a layer having a low index of reflection. The number of the layers and a thickness of each layer are selected to provide designed transmission and blocking characteristics to block circadian-active spectra to be transmitted through the spectral filter. The designed transmission and blocking characteristics include a contiguous blocking region within 455-495 nm band of wavelengths to within at least ±5, and two contiguous transmission regions, a first one of the contiguous regions extending below 455 nm and a second one of the contiguous transmission regions extending above 495 nm. The spectral filter is configured to block 98-100% of the spectral content in the contiguous blocking region, and transmit 80%-100% of the spectral content in the contiguous transmission regions.

In one example embodiment of the spectral filter, the contiguous blocking region extends from 455 nm to 495 nm, the first contiguous transmission region extends from 300 nm to 455 nm, and the second contiguous transmission region extends from 495 nm to at least 700 nm, all with a ±5 nm tolerance, and each layer with the high index of refraction includes titanium dioxide (TiO₂) and has a 2.35 index of refraction, each layer with the low index of refraction includes silicon dioxide (SiO₂) and has a 1.45 index of refraction, and the multi-layer stack includes 81 layers. In another example embodiment of the spectral filter, the contiguous blocking region extends from 455 nm to 495 nm, the first contiguous transmission region extends from 300 nm to 455 nm, and the second contiguous transmission region extends from 495 nm to at least 700 nm, all with a ±2 nm tolerance, and each layer with the high index of refraction includes titanium dioxide (TiO₂) and has a 2.35 index of refraction, each layer with the low index of refraction includes silicon dioxide (SiO₂) and has a 1.45 index of refraction, and the multi-layer stack includes 121 layers.

In still another example embodiment, the filter is configured to receive input illumination from one or more light sources including an atmospheric light source, a light emitting diode (LED), a halogen lamp, or a fluorescent lamp. In yet another example embodiment, the spectral filter does not include a dye-based or a pigment-based material.

The foregoing description of embodiments has been presented for purposes of illustration and description. The foregoing description is not intended to be exhaustive or to limit embodiments of the present invention to the precise form disclosed, and modifications and variations are possible in light of the above teachings or may be acquired from practice of various embodiments. The embodiments discussed herein were chosen and described in order to explain the principles and the nature of various embodiments and its practical application to enable one skilled in the art to utilize the present invention in various embodiments and with various modifications as are suited to the particular use contemplated. While operations are depicted in the drawings in a particular order, this should not be understood as requiring that such operations be performed in the particular order shown or in sequential order, or that all illustrated operations be performed, to achieve desirable results. The features of the embodiments described herein may be combined in all possible combinations of methods, apparatus, modules, and systems.

Only a few implementations and examples are described, and other implementations, enhancements and variations can be made based on what is described and illustrated in this patent document.

I/We claim:

1. A wearable device, comprising:
one or more windows positioned to allow light from a light source to propagate toward a position of a wearer's eyes; and
a spectral filter that comprises a coating positioned on one or more sections of the one or more windows,
wherein the spectral filter includes a multi-layer stack of dielectric material with alternate high and low indices of refraction such that a layer having a high index of refraction is positioned above or below a layer having a low index of reflection, and a layer having a high index of refraction is positioned above or below a layer having a low index of reflection,
wherein a number of the layers and a thickness of each layer are selected to provide designed transmission and blocking characteristics to block circadian-active spectra while allowing spectral content outside of the circadian-active spectra to pass through the spectral filter,
wherein the designed transmission and blocking characteristics include a contiguous blocking region within 455-495 nm band of wavelengths with a tolerance to within at least ±5 nm, and two contiguous transmission regions, a first one of the contiguous regions extending below 455 nm and a second one of the contiguous transmission regions extending above 495 nm, and
wherein the spectral filter is configured to block 98-100% of the spectral content in the contiguous blocking region, and transmit 80%-100% of the spectral content in the contiguous transmission regions.

2. The wearable device of claim 1, wherein the contiguous blocking region extends from 455 nm to 495 nm, the first contiguous transmission region extends from 300 nm to 455 nm, and the second contiguous transmission region extends from 495 nm to at least 700 nm, all with a ±5 nm tolerance.

3. The wearable device of claim 2, wherein each layer with the high index of refraction includes titanium dioxide (TiO₂) and has a 2.35 index of refraction, each layer with the low index of refraction includes silicon dioxide (SiO₂) and has a 1.45 index of refraction, and the multi-layer stack includes 81 layers.

4. The wearable device of claim 1, wherein the contiguous blocking region extends from 455 nm to 495 nm, the first contiguous transmission region extends from 300 nm to 455 nm, and the second contiguous transmission region extends from 495 nm to at least 700 nm, all with a ±2 nm tolerance.

5. The wearable device of claim 4, wherein each layer with the high index of refraction includes titanium dioxide (TiO₂) and has a 2.35 index of refraction, each layer with the low index of refraction includes silicon dioxide (SiO₂) and has a 1.45 index of refraction, and the multi-layer stack includes 121 layers.

6. The wearable device of claim 1, wherein the one or more windows include two lenses, and the spectral filter is formed as the coating on each of the lenses.

7. The wearable device of claim 1, wherein the wearable device is a pair of goggles, the one or more windows forms a unitary window, and the spectral filter is formed as the coating the unitary window.

8. The wearable device of claim 1, wherein the wearable device is a pair of goggles, the one or more windows forms a unitary window, and the spectral filter is formed as the coating on the two or more sections of the unitary window.

9. The wearable device of claim 1, wherein the one or more windows are made of glass or plastic.

10. The wearable device of claim 1, wherein the spectral filter is removably attached to the one or more windows.

11. The wearable device of claim 1, the light source is one or more of: an atmospheric light source, a light emitting diode (LED), a halogen lamp, or a fluorescent lamp.

12. The wearable device of claim 1, further including an anti-reflection coating positioned on one side of the one or more windows.

13. The wearable device of claim 1, wherein the wearable device is a pair of goggles, the one or more windows form a unitary window, the spectral filter is formed as the coating on the two or more sections of the unitary window, and locations and areal extents of the two or more sections of the unitary window are selected to allow light propagating at normal angles to pass through the spectral filter and reach the position of the wearer's eyes.

14. The wearable device of claim 13, wherein the locations and areal extents of the two or more sections of the unitary window are selected to allow light propagating at inclined angles to pass through the spectral filter and reach the position of the wearer's eyes.

15. The wearable device of claim 13, wherein the goggles include opaque side shields positioned to prevent side illumination from reaching the position of the viewer's eyes.

16. A spectral filter for use in an eyewear for restoring circadian rhythm, comprising:
a multi-layer stack coating on a substrate, the multi-layer stack including a plurality of layers of dielectric material with alternate high and low indices of refraction such that a layer having a high index of refraction is positioned above or below a layer having a low index of reflection, and a layer having a high index of refraction is positioned above or below a layer having a low index of reflection,
wherein a number of the layers and a thickness of each layer are selected to provide designed transmission and blocking characteristics to block circadian-active spectra to be transmitted through the spectral filter,
wherein the designed transmission and blocking characteristics include a contiguous blocking region within 455-495 nm band of wavelengths to within at least ±5, and two contiguous transmission regions, a first one of the contiguous regions extending below 455 nm and a second one of the contiguous transmission regions extending above 495 nm, and
wherein the spectral filter is configured to block 98-100% of the spectral content in the contiguous blocking region, and transmit 80%-100% of the spectral content in the contiguous transmission regions.

17. The spectral filter of claim 16, wherein:
the contiguous blocking region extends from 455 nm to 495 nm, the first contiguous transmission region extends from 300 nm to 455 nm, and the second contiguous transmission region extends from 495 nm to at least 700 nm, all with a ±5 nm tolerance, and
each layer with the high index of refraction includes titanium dioxide (TiO₂) and has a 2.35 index of refraction, each layer with the low index of refraction includes silicon dioxide (SiO₂) and has a 1.45 index of refraction, and the multi-layer stack includes 81 layers.

18. The spectral filter of claim 16, wherein:
the contiguous blocking region extends from 455 nm to 495 nm, the first contiguous transmission region extends from 300 nm to 455 nm, and the second contiguous transmission region extends from 495 nm to at least 700 nm, all with a ±2 nm tolerance, and
each layer with the high index of refraction includes titanium dioxide (TiO₂) and has a 2.35 index of refraction, each layer with the low index of refraction includes silicon dioxide (SiO₂) and has a 1.45 index of refraction, and the multi-layer stack includes 121 layers.

19. The spectral filter of claim 16, configured to receive input illumination from one or more light sources including an atmospheric light source, a light emitting diode (LED), a halogen lamp, or a fluorescent lamp.

20. The spectral filter of claim 16, wherein the spectral filter does not include a dye-based or a pigment-based material.
